(19) United States
(12) Patent Application Publication
(10) Pub. No.: US 2014/0277331 A1
(43) Pub. Date: Sep. 18, 2014

(54) Reducing Recoil in Peripherally-Implanted Scaffolds

(71) Applicant: ABBOTT CARDIOVASCULAR SYSTEMS INC., Santa Clara, CA (US)

(72) Inventors: Michael Huy Ngo, San Jose, CA (US); Benjamyn Serna, Gilroy, CA (US); Mikael Trollsas, San Jose, CA (US); Syed Hossainy, Hayward, CA (US)

(73) Assignee: Abbott Cardiovascular Systems Inc., Santa Clara, CA (US)

(21) Appl. No.: 13/801,691

(22) Filed: Mar. 13, 2013

(51) Int. Cl. A61F 2/958

(52) U.S. Cl. CPC A61F 2/958
USPC 623/1.11

(57) ABSTRACT

A peripherally implanted polymer scaffold having a high degree of recoil is worked to reduce recoil in the scaffold when initially deployed at a target lesion in the body. The scaffold is plastically deformed from a crimped state to an expanded state by a balloon catheter. The scaffold is contained within a sheath to prevent recoil up until the point of use. Before the scaffold is introduced to the body, the restraining sheath is removed from the scaffold.

### FIELD OF THE INVENTION

The present invention relates to balloon-expanded polymeric scaffolds that are intended for peripheral vessels of the body.

### BACKGROUND OF THE INVENTION

This invention relates generally to methods of treatment with radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent and scaffold are examples of such an endoprosthesis. Both stents and scaffolds are generally cylindrically shaped devices that function to hold open and sometimes expand a segment of a blood vessel or other anatomical lumen such as urinary tracts and bile ducts. For purposes of this disclosure, use of the term “stent” verses “scaffold” will, unless indicated otherwise, imply the type(s) of material used to form the load bearing portion of the cylindrically shaped. A “stent” is made from biostable or non-degradable material.

A “scaffold” is made from a biodegradable, bioabsorbable, bioresorbable, or bioerodable polymer. The terms biodegradable, bioabsorbable, bioresorbable, biosoluble or bioerodable refer to the property of a material or stent to degrade, absorb, resorb, or erode away from an implant site. For example, the polymer scaffold described in US2011/0190872 is intended to remain in the body for only a limited period of time. The scaffold is made from a biodegradable or bioerodable polymer. In many treatment applications, the presence of a stent in a body may be necessary for a limited period of time until its intended function of, for example, maintaining vascular patency and/or drug delivery is accomplished. Moreover, it has been shown that biodegradable scaffolds allow for improved healing of the anatomical lumen as compared to metal stents, which may lead to a reduced incidence of late stage thrombosis. In these cases, there is a desire to treat a vessel using a polymer scaffold, in particular a bioerodible polymer scaffold, as opposed to a stent, so that the prosthesis's presence in the vessel is for a limited duration.

Scaffolds can be used in the treatment of atherosclerotic stenosis in blood vessels. “Stenosis” refers to a narrowing or constriction of a bodily passage or orifice. In such treatments, scaffolds reinforce body vessels and prevent vasospasm and acute closure, as well as tack up dissections. Scaffolds also reduce restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Scaffolds are typically implanted by use of a catheter which is inserted at an easily accessible location and then advanced through the vasculature to the deployment site. The scaffold is initially maintained in a radially compressed or collapsed state to enable it to be maneuvered through a body lumen. Once in position, the scaffold is usually deployed actively by the inflation of a balloon about which the scaffold is carried on the catheter. The scaffold is mounted on and crimped to the balloon portion the catheter. The catheter is introduced transluminally with the scaffold mounted on the balloon and the scaffold and balloon are positioned at the location of a lesion. The balloon is then inflated to expand the scaffold to a larger diameter to implant it in the artery at the lesion. An optimal clinical outcome requires correct sizing and deployment of the scaffold.

A scaffold intended for a coronary vessel must satisfy a number of basic, functional requirements. The scaffold must be capable of withstanding structural loads, for example, radial compressive forces, imposed on the scaffold as it supports the walls of a vessel after deployment. Therefore, a scaffold must possess adequate radial strength. After deployment, the scaffold must adequately maintain its size and shape throughout its service life despite the various forces that may come to bear on it. In particular, the scaffold must adequately maintain a vessel at a prescribed diameter for a desired treatment time despite these forces. The treatment time may correspond to the time required for the vessel walls to remodel, after which the scaffold is no longer necessary for the vessel to maintain a desired diameter.

Polymer material considered for use as a polymeric scaffold, e.g. poly(L-lactide) (“PLLA”), poly(L-lactide-co-glycolide) (“PLGA”), poly(D-lactide-co-glycolide) or poly(L-lactide-co-D-lactide) (“PLLA-co-PDLA”) with less than 10% D-lactide, and PLLD/PDLA stereo complex, may be described, through comparison with a metallic material used to form a stent, in some of the following ways. A suitable polymer has a low strength to weight ratio, which means more material is needed to provide an equivalent mechanical property to that of a metal. Therefore, struts must be made thicker and wider to have the required strength for a stent to support lumen walls at a desired radius. The scaffold made from such polymers also tends to be brittle or have limited fracture toughness. The anisotropic and rate-dependent inelastic properties (i.e., strength/stiffness of the material varies depending upon the rate at which the material is deformed) inherent in the material, only compound this complexity in working with a polymer, particularly, bio-absorbable polymer such as PLLA or PLGA.

Stents implanted in coronary arteries are primarily subjected to radial loads, typically cyclic in nature, which are due to the periodic contraction and expansion of vessels as blood is pumped to and from a beating heart. Stents implanted in peripheral blood vessels, or blood vessels outside the coronary arteries, e.g., iliac, femoral, popliteal, renal and subclavian arteries, however, must be capable of sustaining both radial forces and crushing or pinching loads. These stent types are implanted in vessels that are closer to the surface of the body. Because these stents are close to the surface of the body, they are particularly vulnerable to crushing or pinching loads, which can partially or completely collapse the stent and thereby block fluid flow in the vessel.

As compared to a coronary scaffold, which is designed to counteract primarily radial loads, a peripheral scaffold must take into account the significant differences between pinching or crushing loads and radial loads, which is discussed for metal stents in Duerig, Tolomeo, Wholey, Overview of superelastic stent Design, Min Invas Ther & Allied Technol 9(3/4), pp. 235-246 (2000) and Stoeckel, Pelton, Duerig, Self-Expanding Nitinol Stents—Material and Design Considerations, European Radiology (2003). The corresponding crushing and radial stiffness properties of the stent also can vary dramatically. As such, a stent that possesses a certain degree of radial stiffness does not, generally speaking, also indicate the degree of pinching stiffness possessed by the stent. The two stiffness properties are not the same, or even similar.

In addition to crushing loads, scaffolds intended for peripheral vessels, as opposed to coronary scaffolds, experience a quite different time-varying loading, to such an extent that the traditional measure of a stent's fitness for use, i.e., its radial strength/stiffness, is not an accurate measure of whether the peripherally implanted scaffold (“peripheral scaffold”) possesses the time-dependent mechanical properties for providing support within the peripheral vessel for the duration needed. This is because a peripheral scaffold is placed in a significantly different environment from a coronary scaffold. The vessel size is larger. And there is much more movement of the vessel, especially when located close to an appendage. As such, a scaffold intended for a peripheral vessel will need to be able to sustain more complex loading, including a combination of axial, bending, torsional and radial loading. See e.g. Bosiers, M. and Schwartz, L., Development of Bioresorbable Scaffolds for the Superficial Femoral Artery, SFA: CONTEMPORARY ENDOVASCULAR MANAGEMENT (‘Interventions in the SFA” section). These and related challenges facing peripherally implanted stents and scaffolds are also discussed in US2011/0190872 (attorney docket no. 104584.10).

Balloon-expanded scaffolds when plastically deformed to a crimped state, and from their crimped states by balloon inflation can exhibit a high degree of recoil. While some promising methods have been proposed to reduce recoil, there is a continuing need to improve upon these methods for reducing recoil.

### SUMMARY OF THE INVENTION

In response to these needs the invention provides methods for reducing recoil of a scaffold. The methods include applying a dwelling balloon pressure and/or post-dilation to a balloon located within a lumen of a scaffold recently implanted at a vessel site within the body using the same or a different balloon. In one embodiment the scaffold is crimped to a balloon of a balloon catheter and enclosed within a sheath to minimize recoil (leading to an undesired increase in scaffold diameter) prior to introducing the scaffold into a body. The sheath is removed before the scaffold is introduced into the body. After the balloon has expanded the scaffold at the vessel site, the same or different balloon is inflated in such a manner as to reduce or minimize acute and/or longer term recoil (leading to an undesired decrease in scaffold diameter) of the implanted scaffold.

In one embodiment a standard balloon inflation protocol is modified by increasing the dwell time significantly. The dwell time may include maintaining a constant pressure or it may include a gentle or light pulsing of the balloon pressure. The dwell time according to this first method may be 2 min, 5, min, 10 min, between 2-5 min, greater than 2 min or between 5 and 10 min.

Dwell time is less forgiven in coronary than in peripheral where stopping the blood flow up to one hour or longer will cause only numbness. Whereas in coronary, w/o adequate blood flow for that same amount of time might cause myocardial infarction. In coronary balloon expandable stents there can be 30 seconds hold time.

In another embodiment balloon pressure is applied in cycles, which can range from one cycle, two cycles, three cycles or more cycles depending on need or duration of each cycle. Functional forms of the applied balloon pressure may include a step, rectified sine/cosine, and parabolic pressure types of pressure profiles. Each function has the following metrics:
On-time (i.e. the hold time after inflation), off-time (i.e. the time between inflation and next inflation)
Frequency=Cycle/sec (number of ON-OFF sequence per sec; this is the inverse of time period)
Max Height of the step (i.e. max ratio of inflation diameter:reference diameter in case of a step function this max height is same as the constant height)
Shape during the ON-time (the first and second derivatives of the pressure profile can define its shape)
   In addition to a true step function, the shape can be a gradual rise (from start to peak pressure) following a gradual delay from peak pressure. In one embodiment there is a gradual rise and fall to/from peak over a period such that there is no OFF time.
   There can be a rapid rise, followed by a gradual decay, or a gradual fall followed by a rapid rise in pressure, such that there is no OFF time.

Additionally, the initial diameter of the target lesion may be taken into account, in the following fashion:
The target lesion can be pre-dilated with 10-20% inflation ratio before scaffold deployment (this coincided with the 1st cycle)
The target lesion can be pre-dilated to a gentler value 5-10% inflation ratio before scaffold deployment (this coincided with the 1st cycle); and the cycle max height can be adjusted to obtain the final desired diameter of the target lesion.

Rather than perform pre-dilation, direct scaffolding a target lesion may be done without pre-dilation. According to one embodiment, the scaffold diameter is increased via balloon pressure to a greater-than-nominal deployment ratio, i.e., for a 6.5 mm deployment in a 6.0 mm vessel the scaffold is deployed to 7 mm. This greater-than-nominal deployment ratio is adjusted to correct for acute recoil. For example for scaffold bench data showing 10% recoil upon deployment, scaffold will be deployed at 15% overexpansion compared to the reference vessel and deployed without any hold time. The overexpansion will correct for the 10% recoil. Alternatively, upon inflation the balloon may be held at the inflated pressure for 15 sec or longer.

According to one aspect of the invention, a scaffold is first positioned to attain the desired apposition with a diseased vessel wall and with a diameter about equal to or slightly greater than a reference vessel diameter. Then balloon pressure is applied for 2 or more minutes to reduce the acute recoil in the scaffold, the recoil one day after implantation and/or the recoil up until one week after implantation. The 2 or more minutes of additional balloon pressure may be applied as a continuation of the pressure used to initially implant the scaffold at the vessel wall, balloon pressure pulses following implantation or placement, or cycles of pressure according to different pressure profiles. For these profiles the period and profile shape may be varied to suit needs.

Preferably the methods according to the invention are used for a scaffold formed from a tube, crimped to a balloon and plastically deformed to an expanded diameter when being placed in the vessel. However, the methods may also be used for other types of scaffolds intended for peripheral vessels.

According to a first implementation there is a method for reducing recoil of a polymeric scaffold at a site in a peripheral vessel of the body, comprising the steps of: using a balloon disposed within the scaffold, inflating the balloon whereby the scaffold attains an expanded diameter; and after the scaffold has the expanded diameter, applying balloon pressure to the scaffold for more than two minutes.

The first implementation may include some or all of the following features, in any combination thereof: wherein the balloon used to expand the scaffold to the expanded diameter and apply the balloon pressure is the same balloon; wherein the scaffold is made from a tube comprising PLLA; wherein the scaffold is crimped to the balloon; wherein the scaffold's expanded diameter is 250-400% of its crimped diameter; wherein the scaffold is inflated at a rate no greater than 6-8 psi/sec when expanded from a crimped diameter to the expanded diameter; wherein the scaffold is made from PLLA, the scaffold has an expanded diameter of at least 6.5 mm and a crimped diameter less than 3 mm; wherein the balloon pressure is applied for 3-5 minutes; wherein the balloon pressure is applied for 5-10 minutes; wherein the scaffold is implanted in the iliac, femoral, popliteal, renal or subclavian artery; wherein the balloon pressure is applied as a sustained balloon pressure when the scaffold attains the expanded diameter, or the balloon pressure comprises a plurality of cycles of balloon pressure each having a duration of 2 or more minutes; and/or wherein the scaffold is made from a polymer tube, or the scaffold is a braided or woven scaffold comprising a polymer.

According to a second implementation there is a method for implanting a polymeric scaffold in a peripheral vessel, comprising the steps of: removing a restraining sheath from a scaffold, the scaffold being crimped to a balloon of a catheter and the sheath being used to reduce recoil of the scaffold; after removing the sheath, introducing the scaffold into a peripheral vessel of the body including placing the scaffold at a target site of the peripheral vessel; inflating the balloon when the scaffold is located at the target site, whereby the scaffold attains an expanded diameter; and after the scaffold has the expanded diameter, applying balloon pressure to the scaffold to reduce recoil.

The second implementation may include some or all of the following features, in any combination thereof: wherein the scaffold has a crimped diameter to expanded diameter ratio of at least 3:1; wherein when the scaffold is plastically deformed by the balloon when the scaffold attains the expanded diameter; wherein the scaffold is formed from a biaxially expanded tube having a diameter equal to or greater than the expanded diameter; wherein the applying balloon pressure includes applying more than one cycle of balloon pressure according to a pressure profile; wherein the pressure profile is one of a rectified size, parabolic and step pressure profile; wherein the pressure profile includes a plurality of cycles of balloon pressure, wherein a period of balloon inflation during a cycle is 1 min, 2 min, or greater than 2 min in duration; and/or wherein the pressure profile varies balloon pressure between a nominal balloon pressure (Po) and a maximum balloon pressure (P1), wherein the nominal balloon pressure is less than a first pressure used to expand the scaffold to the expanded diameter and the maximum balloon pressure is greater than the first pressure.

According to a third implementation there is a method for reducing the recoil of an implanted polymer scaffold, the scaffold being located at a target site in a peripheral vessel, the scaffold being crimped to a balloon of a catheter, comprising the steps of: inflating the balloon to expand the scaffold to an expanded diameter; holding the balloon in an inflated state for greater than two minutes, between 5 and 10 minutes, 5 minutes or 10 minutes; deflating the balloon; and inflating the balloon a second time to reduce the recoil in the implanted polymer scaffold to less than 10% of the expanded diameter.

According to a fourth implementation there is a system for implanting a peripheral scaffold in a body, comprising: a scaffold-catheter system comprising a scaffold crimped to a balloon, the scaffold-catheter system being adapted for use in a medical procedure whereby the scaffold is delivered to a target site in a peripheral vessel of the body and deployed using the balloon catheter; a package containing the scaffold-catheter system; an indicia disposed on or in the package indicating a date when the scaffold-catheter system was made; and instructions for use (IFU) that indicate whether an action should be taken, e.g., whether a first or second step taken, to account for, or compensate for recoil in the scaffold depending on an indicia, e.g., date of manufacture, indicating the age of the device, and including one or more of a device compliance chart showing a hold time verses months aged of the product and a balloon compliance chart showing a plurality of system diameters accounting for recoil. Alternatively, or in addition thereto the IFU may notice a scaffold average recoil of a stated percentage, e.g., 10% or between 5-8% after ½ hour, one hour, one day or one week following implantation and a suggested hold time or balloon inflation protocol to follow to account for any possible recoil.

According to a fifth implementation there is a system or kit, comprising: a scaffold-catheter system comprising a scaffold crimped to a balloon, the scaffold-catheter system being adapted for use in a medical procedure whereby the scaffold is delivered to a target site in a peripheral vessel of the body and deployed using the balloon catheter; a package containing the scaffold-catheter system; an indicia disposed on or in the package indicating a date when the scaffold-catheter system was made; and instructions for use (IFU) that indicate a first step or a second step should be followed to reduce recoil in the scaffold depending on the date when the scaffold-catheter system was made.

The fourth or fifth implementation may include some or all of the following features, in any combination thereof: wherein the indicia provides a date when the scaffold is ready for use by a medical professional; and/or wherein the first step and the second step are when the scaffold-catheter system was made more than three months prior to a date, a balloon pressure lasting more than 5 minutes or more should be applied to reduce scaffold recoil, and if the scaffold-catheter system was made less than three months prior to the date, a balloon pressure lasting from 2-5 minutes should be applied to reduce scaffold recoil; and/or the IFU is provided over a network or with the scaffold-catheter product.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a flow diagram illustrating a first method for reducing recoil in a peripherally implanted scaffold.

FIG. 2 is a flow diagram illustrating a second method for reducing recoil in a peripherally implanted scaffold.

FIG. 3 is a first type of pressure profile to use when reducing recoil in the scaffold according to the second method.

FIG. 4 is a second type of pressure profile to use when reducing recoil in the scaffold according to the second method.

FIG. 5 is a third type of pressure profile to use when reducing recoil in the scaffold according to the second method.

FIG. 6 is a graph showing a reduction in recoil for a scaffold expanded from a balloon according to the second method for reducing recoil.

FIG. 7 is a graph showing a reduction in recoil for a scaffold expanded from a balloon according to the first method for reducing recoil.

### DETAILED DESCRIPTION OF EMBODIMENTS

For purposes of this disclosure, the following terms and definitions apply:

“Reference vessel diameter” (RVD) is the diameter of a vessel in areas adjacent to a diseased section of a vessel that appear either normal or only minimally diseased.

“Inflated diameter” or “expanded diameter” refers to the diameter the scaffold attains when its supporting balloon is inflated to expand the scaffold from its crimped configuration to implant the scaffold within a vessel. The inflated diameter may refer to a post-dilation balloon diameter which is beyond the nominal balloon diameter, e.g., a 6.5 mm balloon has about a 7.4 mm post-dilation diameter, or a 6.0 mm balloon has about a 6.5 mm post-dilation diameter. The nominal to post dilation ratios for a balloon may range from 1.05 to 1.15 (i.e., a post-dilation diameter may be 5% to 15% greater than a nominal inflated balloon diameter). The scaffold diameter, after attaining an inflated diameter by balloon pressure, will to some degree decrease in diameter due to recoil effects related primarily to, any or all of, the manner in which the scaffold was fabricated and processed, the scaffold material and the scaffold design.

“Post-dilation diameter” (PDD) of a scaffold refers to the diameter of the scaffold after being increased to its expanded diameter and the balloon removed from the patient's vasculature. The PDD accounts for the effects of recoil. For example, an acute PDD refers to the scaffold diameter that accounts for an acute recoil in the scaffold.

“Recoil” means the response of a material following the plastic/inelastic deformation of the material. When the scaffold is radially deformed well beyond its elastic range and the external pressure (e.g., a balloon pressure on the luminal surface) is removed the scaffold diameter will tend to revert back to its earlier state before the external pressure was applied. Thus, when a scaffold is radially expanded by applied balloon pressure and the balloon removed, the scaffold will tend to return towards the smaller diameter it had, i.e., crimped diameter, before balloon pressure was applied. A scaffold that has a recoil of 10% within ½ hour following implantation and an expanded diameter of 6 mm has an acute post-dilation diameter of 5.4 mm. The recoil effect for balloon-expanded scaffolds can occur over a long period of time. Post-implant inspection of scaffolds shows that recoil can increase over a period of about one week following implantation. Unless stated otherwise, when reference is made to “recoil” it is meant to mean recoil along a radial direction (as opposed to axial or along longitudinal direction) of the scaffold.

“Acute Recoil” is defined as the percentage decrease in scaffold diameter within the first about ½ hour following implantation within a vessel.

The glass transition temperature (referred to herein as “Tg”) is the temperature at which the amorphous domains of a polymer change from a brittle vitreous state to a solid deformable or ductile state at atmospheric pressure. In other words, Tg corresponds to the temperature where the onset of segmental motion in the chains of the polymer occurs. Tg of a given polymer can be dependent on the heating rate and can be influenced by the thermal history of the polymer. Furthermore, the chemical structure of the polymer heavily influences the glass transition by affecting mobility of polymer chains.

“Stress” refers to force per unit area, as in the force acting through a small area within a plane within a subject material. Stress can be divided into components, normal and parallel to the plane, called normal stress and shear stress, respectively. Tensile stress, for example, is a normal component of stress that leads to expansion (increase in length) of the subject material. In addition, compressive stress is a normal component of stress resulting in compaction (decrease in length) of the subject material.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Modulus” may be defined as the ratio of a component of stress or force per unit area applied to a material divided by the strain along an axis of applied force that result from the applied force. For example, a material has both a tensile and a compressive modulus.

“Toughness”, or “fracture toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough is said to be brittle. Brittle materials are strong, but cannot deform very much before breaking.

As used herein, the terms “axial” and “longitudinal” are used interchangeably and refer to a direction, orientation, or line that is parallel or substantially parallel to the central axis of a scaffold or the central axis of a tubular construct. The term “circumferential” refers to the direction along a circumference of the scaffold or tubular construct. The term “radial” refers to a direction, orientation, or line that is perpendicular or substantially perpendicular to the central axis of the scaffold or the central axis of a tubular construct and is sometimes used to describe a circumferential property, i.e radial strength.

The term “crush recovery” is used to describe how the scaffold recovers from a pinch or crush load, while the term “crush resistance” is used to describe the force required to cause a permanent deformation of a scaffold. A scaffold or stent that does not possess good crush recovery does not substantially return to its original diameter following removal of a crushing force. As noted earlier, a scaffold or stent having a desired radial force can have an unacceptable crush recovery. And a scaffold or stent having a desired crush recovery can have an unacceptable radial force. Crush recovery and crush resistance aspects of peripherally-implanted scaffolds is described in greater detail in US20110190871.

An important factor in scaffold deployment is the rate at which the scaffold is expanded from a crimped state on the balloon to a fully expanded state (crimping to a balloon is described in US2012/0042501, attorney docket no. 62571.448). Inflation of the balloon, which increases the scaffold diameter, is usually achieved through manual inflation/deflation devices that possess a capability for controlled inflation and deflation. The scaffold is plastically deformed, or undergoes an inelastic deformation from a crimped diameter to larger diameter when the balloon is inflated.

The rate of balloon inflation when plastically deforming a polymer scaffold to an expanded diameter within a vessel must not be too fast as this can cause failure in the polymer load bearing structure, e.g., fracture or crack propagation in struts. As noted earlier, unlike a metal, a polymer's stress-strain behavior can be significantly dependent on the rate at which the material undergoes strain, i.e., the strain rate. A crimped scaffold deployed quickly from a balloon can therefore be at a greater risk of being damaged than the same scaffold deployed more slowly. Thus, it can be necessary to increase a scaffold diameter far more slowly than for a metal stent. U.S. application Ser. No. 13/471,263 (attorney docket no. 62571.629) discusses these differences between a polymeric and metal stent and introduces a flow regulator for controlling the rate of balloon inflation. Examples of inflation rates found suitable for expansion of scaffolds from balloons include 6 psi/sec or 2 atm/5 secs, as described in the Instruction For Use (IFU) deployment procedure section for ABSORB BVS and a V59 scaffold delivery system.

An example of a balloon-expanded scaffold intended for being deployed from a balloon according to the disclosure is described in U.S. application Ser. No. 13/549,366 (attorney docket no. 104584.45). The two scaffold patterns, ring, strut and link dimensions and structural characteristics as described in FIGS. 1-6 and the accompanying paragraphs in U.S. application Ser. No. 13/549,366 are each formed from a poly(L-lactide) (“PLLA”) tube in the preferred embodiments. The process for forming a PLLA tube may be the process described in U.S. patent application Ser. No. 12/558,105 (docket no. 62571.382) or US-20120073733 (attorney docket no. 104584.14). Reference is made to a precursor that is “deformed” in order to produce the tube of FIG. 1 of U.S. application Ser. No. 13/549,366 having the desired scaffold diameter, thickness and material properties as set forth therein. Before the tube is deformed or, in some embodiments, expanded to produce the desired properties in the starting tube for the scaffold, the precursor is formed. The precursor may be formed by an extrusion process which starts with raw PLLA resin material heated above the melt temperature of the polymer which is then extruded through a die. Then, in one example, an expansion process for forming an expanded PLLA tube includes heating a PLLA precursor above the PLLA glass transition temperature (i.e., 60-70 degrees C.) but below the melt temperature (165-175 degrees C.), e.g., around 110-120 degrees C. A precursor tube is deformed in radial and axial directions by a blow molding process wherein deformation occurs progressively at a predetermined longitudinal speed along the longitudinal axis of the tube. The deformation improves the mechanical properties of the tube before it is formed into the scaffold of FIGS. 2-4 of U.S. application Ser. No. 13/549,366. The tube deformation process is intended to orient polymer chains in radial and/or biaxial directions. The orientation or deformation causing re-alignment is performed according to a precise selection of processing parameters, e.g. pressure, heat (i.e., temperature), deformation rate, to affect material crystallinity and type of crystalline formation during the deformation process. In an alternative embodiment the tube may be made of poly(L-lactide-co-glycolide), poly(D-lactide-co-glycolide) (“PLGA”), polycaprolactone, (“PCL), any semi-crystalline copolymers combining any of these monomers, or any blends of these polymers. Material choices for the scaffold should take into consideration the complex loading environment associated with many peripheral vessel locations, particularly those located close to limbs. Examples are described in U.S. patent application Ser. No. 13/525,145 (docket no. 104584.43).

Scaffold fabrication processes often form the scaffold from an expanded tube having the same or greater diameter than the expanded diameter of the scaffold. The forming of the tube at these diameters has been desired to impart circumferential polymer chain alignment for radial stiffness at the expanded diameter. Forming the scaffold at this diameter, however, also makes the crimping process more challenging since there is a greater diameter reduction requirement in order to obtain the desired crossing profile for the assembled scaffold-catheter system. Crimping of the scaffold, as detailed in U.S. application Ser. No. 13/194,162 (docket no. 104584.19), may include heating the polymer material to a temperature less then, but near to the glass transition temperature of the polymer. In one embodiment the temperature of the scaffold during crimping is raised to about 5 to 10 degrees below the glass transition temperature for PLLA. When crimped to the final, crimped diameter, the crimping jaws are held at the final crimp diameter for final dwell period. This method for crimping a polymer scaffold having crush recovery is advantageous to reduce recoil when the crimp jaws are released. After the final dwell period, the scaffold is removed from the crimper and a constraining sheath is immediately placed over the scaffold to minimize recoil.

The need to reduce recoil when the scaffold is crimped to the balloon, i.e., recoil outwardly to a larger diameter, is also present when the scaffold is then expanded at the target lesion, i.e., recoil inwardly towards a smaller diameter. The degree of recoil expected for a particular scaffold formed from a tube can depend on several factors, including:
a) The ratio of crimped diameter to expanded diameter. When the scaffold crimped diameter is very small compared to its expanded diameter, e.g., 4:1, 3:1, 5:1, then more recoil is expected than for the same scaffold having less than a 3:1 ratio of these diameters.
b) The ratio of tube diameter to scaffold expanded diameter. When the scaffold as-lased diameter is larger compared to its expanded diameter, e.g., 1.5:1, 1.3:1, 1:1, then less recoil is expected than for the same scaffold having less than a 1:1 ratio of these diameters.
c) The ratio of axial to biaxial expansion during tube formation, the processing parameters used during tube formation, and the material used.
d) The radial stiffness of the scaffold or the particular relationship among expanded diameter, wall thickness, number of crowns, strut width, etc.
e) The amount of time elapsed from when the scaffold was crimped to the balloon to when the scaffold is deployed within the vessel, i.e., the age of the crimped scaffold.

Thus, for example, a scaffold that is expanded to four times its crimped diameter after being restrained within a sheath for six months is expected to have a much higher degree of recoil when balloon expanded than the same scaffold that has been in a sheath for only a few weeks and is expanded to only three times its crimped diameter.

TABLE 1
Recoil percentage for various peripheral scaffolds with compatible
6Fr crimped diameter of 2.03 mm and compatible 7Fr crimped
diameter of 2.33 mm. All samples were expanded to an initial
outer diameter of 5.4 mm and subsequently re-measured for recoil
after 60 minutes.

| Scaffold Type | no aging | 1-month aged | 3-month aged |
|---|---|---|---|
| V79 crimped to 6F/2.03 | 6.5 +/− 1.2% | 11.3 +/− 2.1% | 10.6 +/− 1.8% |
| mm diameter and formed | | | |
| from 7.0 mm tube | | | |
| V79 crimped to 7F/2.33 | 6.7 +/− 1.0% | 7.0 +/− 0.7% | 8.0 +/− 0.7% |
| mm diameter and formed | | | |
| from 7.0 mm tube | | | |
| V80 crimped to 6F/2.03 | 8.3 +/− 1.1% | 9.6 +/− 1.7% | 13.2 +/− 1.6% |
| mm diameter and formed | | | |
| from 7.0 mm tube | | | |
| V80 crimped to 7F/2.33 | 8.0 +/− 0.6% | 8.3 +/− 0.7% | 11.0 +/− 1.4% |
| mm diameter and formed | | | |
| from 7.0 mm tube | | | |
| V79 crimped to 6F/2.03 | 8.3 +/− 0.6% | 10.7 +/− 0.9% | 13.2 +/− 1.3% |
| mm diameter and formed | | | |
| from 6.0 mm tube | | | |

The above data was collected from a study conducted for several scaffolds that were crimped, aged then balloon-expanded according to standard operating procedures for the stent-catheter system. The procedure consists of scaffolds deployed inside a rigid cylindrical tube (5.4 mm ID), which is positioned in water maintained at 37° C. Following deployment, the scaffold's outer diameters are measured and recorded. The scaffolds are then transferred to a 60 ml vial containing water maintained at 37° C. The scaffolds remain in this environment until the required 60 minute time period is achieved. TABLE 1 provides examples of the effects of aging of the crimped scaffold, the effects on recoil when the scaffold is crimped to a smaller diameter and for a smaller tube diameter.

For each of the trials from which the above statistics were computed, the scaffold was crimped to a 5.0 mm balloon, which was then inflated to 5.4 mm. For the “no aging” the scaffold was deployed within a week of crimping. For the “1-month aged” and “3-month aged” cases the scaffold was deployed one-month and three-months after crimping. As expected recoil was worst for the 3-month aged cases and for scaffolds crimped to smaller crimped diameters (2.03 mm verses 2.33 mm). It is also seen that the V79 type of scaffold had slightly less recoil than the V80.

Material sufficiently worked, i.e., subjected to repeated loading/unloading can reduce the effects of recoil. Alternatively, material subject to a constant loading over a prolonged time period can reduce recoil in a scaffold. In the case of a coronary scaffold it is well understood that such techniques for eliminating or reducing recoil are limited, if helpful at all, since a balloon cannot stay inflated or reside within a coronary artery for a prolonged period of time with introducing serious health risks to the patient. As such, methods according to the disclosure, as described in more detail below, generally are not appropriate for coronary-implanted scaffolds or stents.

For peripherally-implanted scaffolds a balloon may stay inflated at the target lesion for an extended period of time, e.g., 10 minutes, without introducing significant health risks to the patient. It is therefore contemplated that a delivery balloon, or subsequently introduced dilatation balloon, may be used to provide an effective means for reducing recoil for a peripherally implanted scaffold by working the scaffold material in its expanded state. By reducing recoil to within acceptable levels, e.g., less than 10% recoil, optimal apposition with the vessel wall over the first week following implantation is more likely to occur.

FIGS. 1-2 depict schematically via flow diagrams medical procedures including methods for reducing recoil in an implanted scaffold using balloon pressure. In the preferred embodiments the methods include the step of removing a sheath that prevents recoil prior to introducing the scaffold into a patient. The sheath was placed on the scaffold immediately after crimping the scaffold to the delivery balloon, to serve one or both of the following purposes—maintain a low crossing profile and high scaffold retention. Without the sheath over the scaffold up until the point of the medical procedure the scaffold is prone to recoil. This need for limiting recoil may be regarded as a byproduct of the pre-crimp diameter to crimped diameter change. As noted above scaffolds may be formed having a tube diameter to crimped diameter ratio of between and including one or more of 2.5:1, 3:1, 4:1, 5:1. After removing the sheath, the scaffold is introduced over a guide wire and located at the target lesion.

Referring to FIG. 1, a first method for reducing recoil using balloon pressure is shown. Once at the target lesion and positioned using balloon markers the balloon is inflated. As noted above, the rate-dependent viscoelastic material of the polymer may require a relatively slow inflation of the balloon. This inflation rate from the crimped state may also be non-constant, as it is believed that the propensity for fracture or failure of struts is more likely during the initial stages of balloon inflation, as described in U.S. application Ser. No. 13/471,263 (attorney docket no. 62571.629). In preferred embodiments inflation of the balloon may proceed according to an average inflation rate of 6 psi/sec or 2/5 atm per second. More generally, it is believed that an inflation rate or deflated to fully inflated (nominal) period should occur over 30 seconds, should take at least 20 seconds, or between 20-30 seconds to ensure that a strain rate in the plastically deforming material does produce excessive brittle behavior as balloon pressure is being increased.

Once reaching the expanded diameter and the scaffold is fully deployed at the target lesion, balloon pressure is maintained for a dwell period to reduce recoil of the scaffold after the balloon is deflated and removed from the target lesion. After the dwell period ends, balloon pressure is reduced and the catheter is removed from the target lesion according to standard operating procedures for the catheter system.

FIG. 7 is a plot showing a significant reduction in recoil when the first method for reducing recoil (FIG. 1) is employed. The scaffold used to generate the plots was V79 which was aged (i.e., crimped to a balloon and placed in a restraining sheath) two months prior to inflation. The catheter system used was the FoxSV™ PTA catheter available from Abbott Vascular in Santa Clara, Calif. The comparison in FIG. 7 is among a 2 min, 5 min and 10 min dwell time. Dwell time is the amount of time in which the balloon is maintained at an approximate constant pressure. In the case of FIG. 7 the dwell pressure is the balloon pressure that inflates the balloon system to its nominal diameter of 5.0 mm. The scaffold/balloon system is inflated to this pressure, into a vessel with an inner diameter of 5.4 mm.

FIG. 7 plots the amount of recoil observed ½ hour after the balloon is removed, three hours after the balloon is removed, and three days after the balloon is removed. As can be seen in FIG. 7, there is a great reduction in acute, three hour and three day recoil when the dwell time is increased from 2 min to 5 min, but comparatively less change when the dwell is increased from 5 min to 10 min.

According to a first aspect of the disclosure, the first method for reducing recoil includes the step of maintaining an inflated balloon state for 5 min, 2-5 min, 3-5 min, 10 minutes, between 5 and 10 minutes, and for more than a 2 minute dwell time. The inflated balloon state may be a nominal balloon diameter, e.g., 6.0 mm for a 6.0 mm balloon, or an overinflated state, e.g., 6.5 mm for a 6.0 mm balloon or the scaffold diameter may be increased from its expanded diameter to a higher diameter during the dwell using a first balloon, e.g., the delivery balloon or a second balloon. The second balloon may have a higher nominal inflated diameter than that of the delivery balloon.

Referring to FIG. 2, a second method for reducing recoil using balloon pressure is shown. The second method may be thought of as a post-dilatation method for reducing recoil. This method, unlike the first method, performs periodic pulses or variations in balloon pressure to work the scaffold material, as opposed to performing an extended dwell time when the scaffold is initially inflated. That is, according to this method balloon pressure is re-applied above a nominal pressure to reduce recoil in the balloon. In FIG. 2 the three-step process (A), (B) and (C) may be repeated one or more times as desired to work the scaffold material. Selection of the amount of cycling or working of the material may be chosen based on a particular scaffold's propensity for recoil or age of the scaffold. The suggested number of repetitions may be proscribed in an IFU for the scaffold-catheter system in terms of the minimum number of cycles to perform to assure that recoil will be within tolerable limits.

Referring again to the second method (FIG. 2), after the initial positioning of the scaffold against the vessel wall, balloon pressure may be reduced or maintained at a nominal working pressure (Po), which may be the nominal balloon pressure (i.e., 6.0 mm balloon diameter for a 6.0 mm balloon), or 5-10%, 10-20%, 10%, 15%, or 20% below the nominal balloon pressure. Po may also be a neutral pressure in the balloon or negative pressure state. Step (A) raises Po over a prescribed time period and rate, which may be constant or non-constant, until the balloon pressure reaches a maximum working pressure. The maximum working pressure (P1) may correspond to the maximum safe pressurization of the balloon or diameter of the scaffold. P1 may be 5-10%, 10-20%, 10%, 15%, or 20% higher than Po or the nominal balloon pressure.

In step (B) the pressure is held for a predetermined time period, e.g., 2 min. This time period will hereinafter be referred to as an “on time” or t-on. In step (C) the balloon pressure is returned to Po. One advantage of using the second method over the first method is that blood flow can be periodically resumed between cycles (i.e., during the off-times).

FIG. 6 shows results from tests conducted using the second method for reducing scaffold recoil. In this example the V79 scaffold (aged 10 months) crimped onto a 6.0 mm balloon and expanded into a 6.4 mm cylindrical vessel is used. Recoil was measured over the first ½ hour (acute), 1 hour, 24 hour and 6 days after implantation. The recoil shows a comparison between a single 2 min dwell, two cycles, i.e., two 2 min dwells, and three 2 min dwells. As can be seen, there is a consistent reduction in recoil for each additional cycle according to the second method (FIG. 2). In these tests the on-time or t-on is 2 min.

FIGS. 3-5 provide examples of balloon pressure profiles for use with the second method. Specifically, these pressure profiles for steps (A), (B) and (C) may be practiced by applying these pressures over time (as illustrated) to the balloon, where one cycle of steps (A), (B) and (C) from FIG. 2 occur over the illustrated periods T10, T20 and T30, respectively. Thus in each of FIGS. 3-5 there are three cycles of steps (A), (B) and (C) shown. Balloon inflation devices capable of providing the pressure profiles illustrated in FIGS. 3-5 may be found in, or taught by U.S. application Ser. No. 13/471,263 (attorney docket no. 62571.629), U.S. application Ser. No. 13/436,527 (attorney docket no. 62571.620) and U.S. Pat. No. 6,419,657.

Referring to FIG. 3, there are three cycles of pressure profiles 10, 14 and 16. The pressure profile verses time has a relatively fast rise time 11, e.g., 5-10 seconds, followed by the t-on period at P1, e.g., 2 min, then a similar drop time 12 where the pressure returns to Po. The period for one cycle, i.e., steps (A), (B) and (C) in FIG. 2, is T10. The pressure profile described in FIG. 3 defines a “step-function” or “step” type of pressure profile for working the scaffold between P1 and Po to reduce recoil.

Referring to FIG. 4, there are three cycles of pressure profiles 20, 24 and 26. For step (A) the pressure profile verses time is not constant, rising initially quickly then slowing as the pressure reaches P1. For Step (B) the on-time is pressure within +/−5% of P1, e.g., 2 min or 1 min, followed by an abrupt drop in pressure 22. The pressure profile described in FIG. 4 defines a “parabolic” type of pressure profile for working the scaffold between P1 and Po to reduce recoil, since the rise time 21 resembles a parabolic curve. The period for one cycle, i.e., steps (A), (B) and (C) in FIG. 2, is T20. The pressure profile about the +/−5% of P1 peak is asymmetric for the “parabolic” pressure profile, but symmetric for the “step” pressure profile.

Referring to FIG. 5, there are three cycles of pressure profiles 30, 34 and 36. For step (A) the pressure profile verses time is not constant, rising initially quickly then slowing as the pressure reaches P1. For Step (B) the on-time is pressure within +/−5% of P1, e.g., 2 min or 1 min, followed by a same rate of decrease in pressure 32 so that the profile is symmetric about the +/−5% of P1 peak. The pressure profile described in FIG. 5 defines a “rectified sine” type of pressure profile for working the scaffold between Pb and Po to reduce recoil, since the profile resembles a rectified sine waveform. The period for one cycle, i.e., steps (A), (B) and (C) in FIG. 2, is T30.

The periods T10, T20, or T30 for each cycle may be 15 sec, 30 sec, up to a minute, 1 or 2 minutes, 5 minutes, or 2-5 minutes and may vary from cycle to cycle, e.g., later cycles having a shorter period than prior cycles. The on time or t-on may range from 10 sec, 15 sec, 30 sec, 1 min, 2 min, 2-5 min or 5 min.

The choice among step, parabolic and rectified sine may vary according to the type of inflation system being used, a method found more effective over another method for working the material sufficiently by applied external pressure, minimizing the time needed within the patient's vasculature to reduce recoil, and/or the most simple or user-preferred process to implement based on past practices. Additionally, the choice may lie in the type of inflation system preferred. For example, the parabolic waveform may be preferred for cycling the pressure when an inflation valve permits a relatively abrupt drop in pressure but a controlled increase in pressure. An example of such an inflation device is found in U.S. Pat. No. 6,419,657. Similarly, when using a device that allows only a controlled, slow inflation and deflation the rectified sine or step may instead be employed. Additionally, it is contemplated that a more effective working of the material may occur using three rectified sine pressure profiles over one or two step functions because the more rapid working of the material is found more effective in reducing recoil for a particular scaffold design. One approach over another may also be chosen based on the type of lesion being treated or difficulties that may be encountered in achieving/maintaining an optimal apposition.

In other embodiments of the first and second methods the scaffold may not have a restraining sheath, or may include a sheath restraining it to a balloon when the scaffold is being delivered to the vessel site. The methods are, moreover, not limited to scaffolds formed from a balloon. Instead, it is contemplated that the methods may also provide benefits for minimizing recoil effects present in braided or woven polymeric scaffolds. In general, the method is applicable for any polymeric device that goes through the process of crimping where stress relaxation occurs as it ages.

According to the disclosure, some or all of the steps described in FIG. 1 or 2 may be embodied in, e.g., an Instructions for Use (IFU) for a peripheral scaffold-catheter system. The IFU may be included with the scaffold-catheter system received by a medical professional, or otherwise provided to a medical professional, e.g., via a network address. An example of an IFU is the Armada™ Percutaneous Transluminal Angioplasty Catheter IFU available from “http://www.abbottvascular.com/us/ifu.html”.

The scaffold-catheter packaging may include an assembly date, or indicia indicating when the scaffold-catheter system was made, such as the date when the scaffold was crimped to the balloon or when the package was sterilized and initially made ready for use by a medical professional. This information may be informative as an indicator of the possible ranges of recoil that might occur after the scaffold is implanted. With this information the IFU may include recommendation(s) of the post-dilation procedure to use to reduce recoil based on the age of the scaffold-catheter system. For example, with respect to both the first and second methods, the amount of cycling (or duration of the dwell time for either the first or second method) may be prescribed based on the aging of the scaffold, which can be indicated on the packaging. For example, if the scaffold was placed within the sheath 1, 2, 3, or 4 months prior to its use (as indicated by a date stamp, color coding or other suitable indicia indicating its age, then the IFU may prescribe 1, 2, 3, or 4 cycles under the second method (FIG. 2), or a dwell period of 4, 5, 6 or 7 minutes under the first method (FIG. 1).

According to one embodiment the device's IFU includes a device compliance chart. Presently, a balloon compliance chart is included with an IFU, an example of which is reproduced below as TABLE 2. Here is a balloon compliance chart for a 6.0 mm balloon having a rated burst pressure of 14 atm.

TABLE 2

| | | Scaffold ID (mm) |
|---|---|---|
| Pressure | Pressure | by system |
| (atm) | (kPa) | diameter (mm) |
| 8 | 811 | 6.01 |
| 9 | 912 | 6.09 |
| 10 | 1013 | 6.15 |
| 11 | 1115 | 6.21 |
| 12 | 1216 | 6.25 |
| 13 | 1317 | 6.29 |
| 14 (RBP) | 1419 | 6.33 |
| 15 | 1520 | 6.36 |
| 16 | 1621 | 6.38 |
| 17 | 1723 | 6.40 |

TABLE 3 shows one example of a device compliance chart, in accordance with the disclosure.

TABLE 3

[chemical structure]

This chart may be included within an IFU to provide suggestions for a minimum balloon pressure hold time, or duration of balloon pressure cycling based on the months aged of the scaffold-catheter assembly. The shaded blocks indicate the number of minutes hold time suggested. For a product aged 8 months the minimum recommended hold time, or duration of balloon pressure cycling could be 6 minutes, whereas if the aged time is 2 months the minimum duration could be only 1 minute. Since a longer hold time should not cause problems, and since a balloon may remain inflated within a peripheral vessel, thereby obstructing blood flow (or using method 2 periodically resumed between inflation on-times) the hold time for a 1 or 2 month aged product may be from 1 to 7 minutes.

With regard to TABLES 2 and 3, a medical professional would utilize the TABLE 2 chart for pressure to obtain the desired diameter and the TABLE 3 chart for the duration hold-time based on the aging of the product. Additionally, the charts or IFU may note that the hold times are intended to target a specific recoil percentage (10%, 8% or less than 10%).

In another embodiment TABLE 2 may be represented as adjusted values reflecting the recoil of the scaffold when expanded to the system diameters, as shown, but accounting for recoil, e.g., 10% recoil of the scaffold. In this embodiment there may be only a TABLE 2 part of the IFU (no TABLE 3). The TABLE 2 of this embodiment includes system diameters that account for recoil and a notation or notice that the system diameters refers to a diameter after a certain time period has elapsed, e.g., ½ hour, one hour, and/or 24 hours after implantation and reflecting an average recoil of 10% for the scaffold.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method for reducing recoil of a polymeric scaffold at a site in a peripheral vessel of the body, comprising the steps of:
using a balloon disposed within the scaffold, inflating the balloon whereby the scaffold attains an expanded diameter; and
after the scaffold has the expanded diameter, applying balloon pressure to the scaffold for more than two minutes.

2. The method of claim 1, wherein the balloon used to expand the scaffold to the expanded diameter and apply the balloon pressure is the same balloon.

3. The method of claim 1, wherein the scaffold is made from a tube comprising PLLA.

4. The method of claim 1, wherein the scaffold is crimped to the balloon.

5. The method of claim 4, wherein the scaffold's expanded diameter is 250-400% of its crimped diameter.

6. The method of claim 4, wherein the scaffold is inflated at a rate no greater than 6-8 psi/sec when expanded from a crimped diameter to the expanded diameter.

7. The method of claim 4, wherein the scaffold is made from PLLA, the scaffold has an expanded diameter of at least 6.5 mm and a crimped diameter less than 3 mm.

8. The method of claim 1, wherein the balloon pressure is applied for 3-5 minutes.

9. The method of claim 1, wherein the balloon pressure is applied for 5-10 minutes.

10. The method of claim 1, wherein the scaffold is implanted in the iliac, femoral, popliteal, renal or subclavian artery.

11. The method of claim 1, wherein the balloon pressure is applied as a sustained balloon pressure when the scaffold attains the expanded diameter, or the balloon pressure comprises a plurality of cycles of balloon pressure each having a duration of 2 or more minutes.

12. The method of claim 1, wherein the scaffold is made from a polymer tube, or the scaffold is a braided or woven scaffold comprising a polymer.

13. A method for implanting a polymeric scaffold in a peripheral vessel, comprising the steps of:
removing a restraining sheath from a scaffold, the scaffold being crimped to a balloon of a catheter and the sheath being used to reduce recoil of the scaffold;
after removing the sheath, introducing the scaffold into a peripheral vessel of the body including placing the scaffold at a target site of the peripheral vessel;
inflating the balloon when the scaffold is located at the target site, whereby the scaffold attains an expanded diameter; and
after the scaffold has the expanded diameter, applying balloon pressure to the scaffold to reduce recoil.

14. The method of claim 13, wherein the scaffold has a crimped diameter to expanded diameter ratio of at least 3:1.

15. The method of claim 13, wherein when the scaffold is plastically deformed by the balloon when the scaffold attains the expanded diameter.

16. The method of claim 13, wherein the scaffold is formed from a biaxially expanded tube having a diameter equal to or greater than the expanded diameter.

17. The method of claim 13, wherein the applying balloon pressure includes applying more than one cycle of balloon pressure according to a pressure profile.

18. The method of claim 17, wherein the pressure profile is one of a rectified size, parabolic and step pressure profile.

19. The method of claim 17, wherein the pressure profile includes a plurality of cycles of balloon pressure, wherein a period of balloon inflation during a cycle is 1 min, 2 min, or greater than 2 min in duration.

20. The method of claim 17, wherein the pressure profile varies balloon pressure between a nominal balloon pressure (Po) and a maximum balloon pressure (P1), wherein the nominal balloon pressure is less than a first pressure used to expand the scaffold to the expanded diameter and the maximum balloon pressure is greater than the first pressure.

21. A method for reducing the recoil of an implanted polymer scaffold, the scaffold being located at a target site in a peripheral vessel, the scaffold being crimped to a balloon of a catheter, comprising the steps of:
inflating the balloon to expand the scaffold to an expanded diameter;
holding the balloon in an inflated state for greater than two minutes, between 5 and 10 minutes, 5 minutes or 10 minutes;
deflating the balloon; and
inflating the balloon a second time to reduce the recoil in the implanted polymer scaffold to less than 10% of the expanded diameter.

22. A kit, comprising:
a scaffold-catheter system comprising a scaffold crimped to a balloon, the scaffold-catheter system being adapted for use in a medical procedure whereby the scaffold is delivered to a target site in a peripheral vessel of the body and deployed using the balloon catheter;
a package containing the scaffold-catheter system;
an indicia disposed on or in the package indicating a date when the scaffold-catheter system was made; and
instructions for use (IFU) that indicate a first step or a second step that should be followed to reduce recoil in the scaffold depending on the date when the scaffold-catheter system was made.

23. The kit of claim 22, wherein the indicia provides a date when the scaffold is ready for use by a medical professional.

24. The kit of claim 22, wherein the first step and the second step are
when the scaffold-catheter system was made more than three months prior to a date, a balloon pressure lasting more than 5 minutes or more should be applied to reduce scaffold recoil, and
if the scaffold-catheter system was made less than three months prior to the date, a balloon pressure lasting from 2-5 minutes should be applied to reduce scaffold recoil.

25. The kit of claim 22, wherein the first step is a first inflation pressure resulting in a first recoiled diameter of the scaffold and the second step is second inflation pressure resulting in a second recoiled diameter of the scaffold.
